(19) United States
(12) Patent Application Publication
(10) Pub. No.: US 2025/0114072 A1
(43) Pub. Date: Apr. 10, 2025

(54) WEARABLE AND BODY-CONFORMABLE ULTRASOUND TRANSDUCER ARRAY FOR MUSCLE IMAGING AND MUSCLE-IN-THE-LOOP CONTROL

(71) Applicant: North Carolina State University, Raleigh, NC (US)

(72) Inventors: Nitin Sharma, Raleigh, NC (US); Xiaoning Jiang, Raleigh, NC (US); Xiangming Xue, Raleigh, NC (US); Sunho Moon, Raleigh, NC (US)

(21) Appl. No.: 18/910,226

(22) Filed: Oct. 9, 2024

Related U.S. Application Data
(60) Provisional application No. 63/543,128, filed on Oct. 9, 2023.

(51) Int. Cl. A61B 8/00; A61B 8/08

(52) U.S. Cl. CPC A61B 8/4494; A61B 8/08; A61B 8/4209; A61B 8/52

(57) ABSTRACT

A wearable ultrasound device may include an array of ultrasound transducers, wherein each ultrasound transducer comprises: a matching layer on a top side, an active layer adjacent the matching layer, a first backing layer adjacent the active layer, a second backing layer adjacent the first backing layer; and an epoxy bonding material interspaced between each of the matching layer, the active layer, the first backing layer, and the second backing layer, wherein the array of ultrasound transducers is surrounded by a PDMS filling, whereby the active layer is configured to communicate a signal in response to mechanical deformation.

### CROSS-REFERENCE TO RELATED APPLICATIONS

This application claims priority U.S. Provisional Patent Application No. 63/543,128, filed Oct. 9, 2023, which is incorporated herein by reference in its entirety.

### STATEMENT REGARDING FEDERALLY SPONSORED RESEARCH OR DEVELOPMENT

This invention was made with government support under grant numbers EB032059 and 2124017 awarded by the National Science Foundation. The government has certain rights in the invention.

### BACKGROUND

The present disclosure relates generally to ultrasound sensors and muscle activity detection.

Existing systems for measuring, detecting, and/or controlling muscle activity include surface electromyography (sEMG). Surface electromyography (sEMG) measures the electrical activity of muscles from the surface of the skin and is one of the principal non-invasive techniques to represent human voluntary motion and movement intention. However, sEMG has some inherent limitations, such as a low signal-to-noise ratio (SNR), an inability to reliably monitor the deeper muscles, the degradation of signal due to fatigue, insufficient ability to distinguish relative muscle firings between adjacent muscles, also known as muscle crosstalk, and the electromechanical delay (EMD), which is defined as the time lag between the onset of electrical activation and the beginning of force production. Several limitations of sEMG can be avoided through implantable electromyography (EMG) and targeted muscle reinnervation strategies. However, these approaches are invasive.

Therefore, a need exists for a muscle activity measuring, detecting, and/or controlling device that is non-invasive and penetrates different depths and small regions of the muscle tissue.

### SUMMARY

According to some implementations, a wearable ultrasound sensor is disclosed, the wearable ultrasound sensor configured to detect muscle activities. The wearable ultrasound sensor includes an array of ultrasound transducers, wherein each ultrasound transducer includes a matching layer on a top side, an active layer adjacent the matching layer, a first backing layer adjacent the active layer, a second backing layer adjacent the first backing layer, and an epoxy bonding material interspaced between each of the matching layer, the active layer, the first backing layer, and the second backing layer. The array of ultrasound transducers is surrounded by a PDMS filling. The active layer is configured to communicate a signal in response to mechanical deformation.

In some implementations, the wearable ultrasound sensor is also configured as a wearable functional electrical stimulation (FES) electrode.

In some implementations, the matching layer includes aluminum oxide and epoxy.

In some implementations, the active layer is a piezo layer including PZT-5A.

In some implementations, the first backing layer includes E-solder 3022 and the second backing layer includes tungsten and epoxy.

In some implementations, the wearable ultrasound sensor further includes a wire connection to each of a top side of the active layer and a bottom side of the active layer.

In some implementations, the array of ultrasound transducers includes 16 ultrasound transducers.

In some implementations, the wearable ultrasound sensor further includes a data acquisition system in electrical communication with each of the ultrasound transducers of the array of ultrasound transducers, the data acquisition system configured to collect data from the array of ultrasound transducers during a muscle activation or muscle detection operation.

In some implementations, the wearable ultrasound sensor further includes a controller in electrical communication with the data acquisition system, the controller configured for processing signals from the array of ultrasound transducers.

In some implementations, the array of ultrasound transducers is configured to be coupled to at least a portion of a muscle grouping of a user.

In some implementations, a thickness of each ultrasound transducer is less than 1.5 mm.

In some implementations, the array of ultrasound transducers has a center frequency of 10 MHz configured to detect muscle movements.

According to some implementations, a system is disclosed. The system includes a wearable ultrasound sensor and a controller. The wearable ultrasound sensor is configured to detect muscle activities. The wearable ultrasound sensor includes an array of ultrasound transducers, wherein each ultrasound transducer includes a matching layer on a top side, an active layer adjacent the matching layer, a first backing layer adjacent the active layer, a second backing layer adjacent the first backing layer, and an epoxy bonding material interspaced between each of the matching layer, the active layer, the first backing layer, and the second backing layer. The array of ultrasound transducers is surrounded by a PDMS filling. The active layer is configured to communicate a signal in response to mechanical deformation. The controller is in electrical communication with the array of ultrasound transducers of the wearable ultrasound sensor. The controller is configured to collect and interpret signals from the array of ultrasound transducers in response to a muscle activity.

In some implementations, the active layer of the wearable ultrasound sensor is a piezo layer including PZT-5A, the active layer configured to send a signal to the controller in response to mechanical deformation.

In some implementations, the wearable ultrasound sensor is also configured as a wearable functional electrical stimulation (FES) electrode, and the controller is configured to send electrical stimulation signals to the array of transducers of the wearable ultrasound sensor.

According to some implementations, a method of fabricating a wearable ultrasound sensor is disclosed. The method includes providing a matching layer, an active layer, a two backing layers. The method further includes stacking the matching layer, the active layer, and the two backing layers using an epoxy to form a bonded stack. The method further includes dicing the bonded stack into a plurality of ultrasound sensors including a first ultrasound sensor. The method further includes coupling a wire to the first ultrasound sensor, the wire configured to relay data from the first ultrasound sensor to a data acquisition system or a controller. The method further includes coating the plurality of ultrasound sensors with a protective material which, when cured, forms a flexible substrate within which the plurality of ultrasound sensors is arranged in an array.

In some implementations, the wearable ultrasound sensor is configured for conforming and attaching to a skin surface of a mammal.

In some implementations, the protective material is liquid PDMS that cures to form the flexible substrate.

In some implementations, the first ultrasound sensor of the plurality of ultrasound sensors has a width less than 1.5 mm and a thickness less than 1.5 mm.

In some implementations, the method further includes providing a controller coupled to and in electrical communication with the plurality of ultrasound sensors, the controller configured to collect muscle movement signals from the plurality of ultrasound sensors.

Additional advantages will be set forth in part in the description that follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1A is an image of an AgNW/PDMS customized FES electrode, according to one implementation.

FIG. 1B is a schematic of the structure of the customized electrode of FIG. 1A, according to one implementation.

FIG. 2A and FIG. 2B show an experimental setup for evaluation of the effectiveness of the customized FES electrode, according to one implementation.

FIG. 2C and FIG. 2D show an experimental setup for a feasibility test of the customized FES electrode, according to one implementation.

FIG. 3 shows graphs of the evaluation of the electrical stimulation effect of the customized FES electrode on the wrist extensor response in comparison to the performance of the commercial FES electrode with different stimulation parameters: current amplitude (5-11 mA), frequency (20-40 Hz), and pulse duty cycle (20-40%) of electrical stimulation, according to various implementations.

FIG. 4A shows B-mode ultrasound imaging of target layers of the muscle (i & ii) as a reference at the status of relaxation and contraction of the wrist extensor.

FIG. 4B shows graphs of A-mode echo signal of the single-element ultrasound transducer from muscle layers i & ii to see if the time of flight (TOF) was matched with layers of muscle presented in B-mode ultrasound imaging.

FIG. 5A shows graphs of peak-to-peak voltage (Vpp) changes measured to see the insertion loss depending on the presence of the customized FES electrode, according to one implementation.

FIG. 5B shows graphs of the echo signals from the layers of the muscle (i & ii) that were collected to compare the effects of the presence of the customized and commercial FES electrode on the front of the ultrasound transducer, according to one implementation.

FIG. 6A shows a schematic demonstration of the wearable ultrasound transducer design, according to one implementation.

FIG. 6B shows a diagram of the fabrication process for the wearable ultrasound transducer, according to one implementation.

FIG. 7A shows an image of an example transducer with an example size of about 1.5 cm, which can be used for single muscle measurements, according to one implementation.

FIG. 7B shows an image of an example transducer with an example size of about 10 cm, which can be used for multiple muscle measurements, according to one implementation.

FIG. 7C shows an image of an example transducer demonstrating the flexibility of the wearable transducer, according to one implementation.

FIG. 8A shows a schematic of the experimental setup for transducer characterization for a pulse/echo test for the transducer, according to one implementation.

FIG. 8B shows schematics of the experimental setups for transducer characterization for a pulse/echo test for electrical impedance measurements for the transducer element in water and in air, according to one implementation.

FIG. 9A shows an experimental setup for an in-vitro test with block diagrams of the experimental setup, including a side view, according to one implementation.

FIG. 9B shows the experimental setup of FIG. 9A with a top view, according to one implementation.

FIG. 9C shows photographs of the experimental setup depicted in FIG. 9A, according to one implementation.

FIG. 10A shows an experimental setup used for the in-vivo test including a block diagram of the experimental system, according to one implementation.

FIG. 10B shows photographs of the experimental setup depicted in FIG. 10A.

FIG. 11A shows a graph of transducer characterization element #10 showing measured electrical impedance, according to one implementation.

FIG. 11B shows a graph of transducer characterization element #10 showing the pulse-echo response of element #10, according to one implementation.

FIG. 12A shows the variations of received RF signals from pork #1 at different pulling distances from T0 to T5 from element #2, according to one implementation.

FIG. 12B shows the variations of received RF signals from pork #1 at different pulling distances from T0 to T5 from element #16, according to one implementation.

FIG. 13A shows a diagram of muscle movement imaging for Pork #2 wherein the grayscale bar represents the relative displacement of the muscle, with T0 as the reference, according to one implementation.

FIG. 13B shows a diagram of muscle movement imaging for Pork #1 wherein the grayscale bar represents the relative displacement of the muscle, with T0 as the reference, according to one implementation.

FIG. 14 shows a diagram of muscle average movement imaging during an in-vivo test, wherein the grayscale bar represents the relative displacement of the muscle, and the displacement was calculated using the forearm at 0° as the reference, according to one implementation.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a wearable ultrasound electrode is shown, according to various implementations.

For background context, ultrasound probes typically include transducer piezoelectric ceramic elements sandwiched between a backing or damping layer and a set of matching layers. The backing layers prevent the backward-emitted sound waves from echoing and ringing back into the transducer for detection. The matching layer or layers provide the required acoustic impedance gradient for the acoustic energy from the transducer to smoothly penetrate the body tissue and for the reflected acoustic waves (the returning echo) to smoothly return to the transducer for detection.

Ultrasound (US) imaging is an effective method for measuring muscle activity. Nevertheless, conventional ultrasound (US) transducers are cumbersome and inflexible, making them inconvenient for continuous monitoring of muscle activity for assistive robotics (AR) control. In light of no report available about using a flexible transducer for detecting muscle activities for AR, a study was conducted that developed a novel wearable US device for detecting muscle activities. Specifically, as disclosed herein, a 16-element 10 MHz flexible sparse array was designed, fabricated, and characterized. The feasibility of monitoring muscle activity in different regions was demonstrated by an in vivo human experiment.

Robotic prostheses and powered exoskeletons are novel assistive robotic devices for modern medicine. Muscle activity sensing plays an important role in controlling assistive robotics devices. Most devices measure the surface electromyography (sEMG) signal for myoelectric control. However, sEMG is an integrated signal from muscle activities. It is difficult to sense muscle movements in specific small regions, particularly at different depths. Alternatively, traditional ultrasound imaging has recently been proposed to monitor muscle activity due to its ability to directly visualize superficial and at-depth muscles. Despite their advantages, traditional ultrasound probes lack wearability.

This study devised a wearable ultrasound (US) transducer, based on lead zirconate titanate (PZT) and a polyimide substrate, for sensing muscle activity. The fabricated PZT-5A elements were arranged into a 4×4 array and then packaged in polydimethylsiloxane (PDMS). In-vitro porcine tissue experiments were carried out by generating the muscle activities artificially, and the muscle movements were detected by the proposed wearable US transducer via muscle movement imaging. Experimental results showed that all 16 elements had very similar acoustic behaviors: the averaged central frequency, −6 dB bandwidth, and electrical impedance in water were 10.59 MHz, 37.69%, and 78.41Ω, respectively. The in vitro study successfully demonstrated the capability of monitoring local muscle activity using the prototyped wearable transducer. The findings indicate that ultrasonic sensing may be an alternative to standardized myoelectric control for rehabilitation robots and functional electrical stimulation.

Traditional ultrasound probes are not designed for closed-loop control. They are not body-conformable and wearable. The ultrasound transducers developed by this study are body-conformable and wearable.

Furthermore, in some implementations, these ultrasound probes implement functional electrical stimulation (FES). FES is a treatment that applies small electrical charges to a muscle that has become paralyzed or weakened (e.g., due to damage to the brain or spinal cord). The electrical charge stimulates the muscle to make its usual movement.

### Example System and Study #1—Evaluation of a Body-Conforming Electrode for Functional Ultrasound Compatible Electrical Stimulation

Abstract—Functional electrical stimulation (FES) is a neurorehabilitation modality that helps improve the size and strength of atrophied muscles after paralysis and reduce spasticity. However, FES causes muscle fatigue, and monitoring the patient's response to avert muscle fatigue caused by FES becomes imperative. Ultrasound (US) imaging can elucidate valuable information about muscle thickness, stiffness, force, and fatigue in the muscles. However, if the FES electrodes do not match the acoustic properties of the surrounding tissues, it could result in interference with other electronic devices, potentially affecting both US imaging and the performance of the FES system. This study evaluates a specially designed body-conforming FES customized electrode that is intended to be compatible with ultrasound. The electrode is made with silver nanowires/Polydimethylsiloxane (AgNW/PDMS). The performance of the body-conforming customized FES electrode was demonstrated in parallel to that of the commercial hydrogel electrode. Moreover, compatibility with ultrasound (US) was established through tests employing a 3.5 MHz single-element US transducer. The customized FES electrode exhibited a comparably minor variance (<8%) relative to the commercial hydrogel electrode.

Discussion—Spinal cord injury (SCI) and stroke are major neurological incidents leading to paralysis. Referring to the Traumatic Spinal Cord Injury facts and figures at a glance 2023 from the National Spinal Cord Injury Statistical Center, the United States witnesses approximately 18,000 new patients diagnosed with SCI annually, and a total of 302,000 people with SCI are estimated to live in the United States. According to the American Stroke Association, stroke is the fifth fatal cause of death and disability in the United States. The physical impairment of one side of the body is a common consequence of stroke. The potential for recovering the mobility of the impaired body usually diminishes six months after having a stroke. Substantially decreased quality of life motivates these individuals to seek rehabilitation interventions that ameliorate the symptoms of paralysis.

Functional electrical stimulation (FES) is a therapeutic application used in neurorehabilitation to facilitate the functional movement of a paralyzed limb. However, it is limited to use FES alone due to the rapid onset of muscle fatigue, resulting in swift loss of FES-elicited muscle force. Sonomyography using ultrasound (US) imaging can evaluate muscle fatigue induced by the FES. Ultrasound imaging can penetrate deep into muscle to detect the activities, unlike other sensing modalities, e.g., surface electromyography (sEMG), which has a low signal-to-noise ratio (SNR) and an inability to monitor deeper muscles reliably. US offers relatively high-resolution imaging and therefore can provide real-time monitoring of muscle activities during FES stimulation. In addition, since US imaging yields information about location rather than force, it cannot induce muscular fatigue, which bestows another benefit to its usage. Given the merits of US imaging, an acoustically matched FES electrode was developed in this study, enabling precise muscle monitoring in the specific area of FES implementation.

Given these considerations, this study designed and fabricated silver nanowires/Polydimethylsiloxane (AgNW/PDMS) electrodes customized for ultrasound compatibility. The inclusion of PDMS serves to mitigate electromagnetic noise that arises from FES without the distortion of US imaging. A validation test was conducted to evaluate the effectiveness of the customized FES electrode by comparing it with commercial hydrogel FES electrodes. By measuring the wrist bend angle caused by wrist extensor stimulation, the performance of both FES electrodes was compared with different stimulation parameters: current amplitude, frequency, and pulse width. Furthermore, a 3.5 MHz single-element ultrasound transducer was used for assessing the feasibility of the ultrasound-compatible AgNW/PDMS customized electrode in this work. Since current commercially available FES electrodes can be a hindrance to on-the-spot monitoring of muscle activities, this strategic integration was applied to validate the electrode's capacity to enable the transmission of ultrasound waves even when functional electrical stimulation (FES) is active, all the while circumventing significant insertion loss.

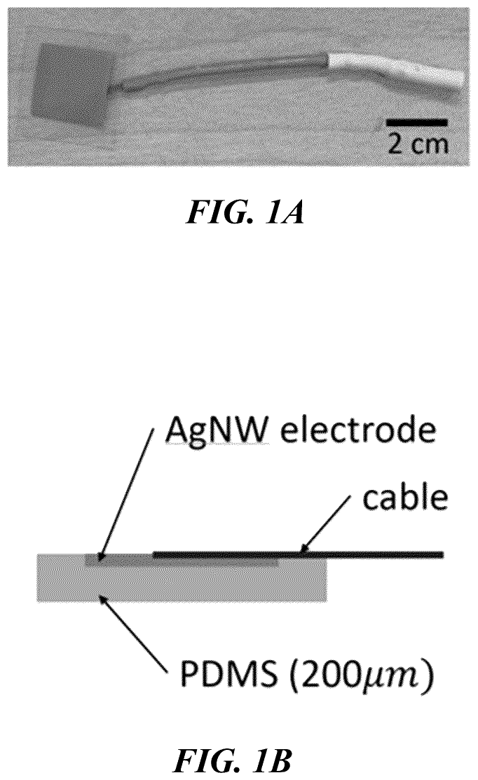

Methods—Ultrasound Compatible FES Electrode Design and Fabrication—Silver nanowires (AgNWs) were synthesized by a modified polyol process [10, 11]. Next, AgNWs were drop-cast onto a precleaned glass slide with Kapton tape as a sacrificial mask. AgNWs were thermal annealed to fuse the AgNW junctions to a uniform and conductive network. The thickness of AgNW film ranged from one to several microns. The resulting AgNW film was patterned using a laser cutter (Universal Laser System VLS 6.60 Laser) to dimensions of 2 cm by 2 cm. Degassed liquid PDMS (sylgard 184, Dow Corning) was spin-coated on the AgNW film at 400 rpm for 30 seconds and cured at 50° C. for 12 h. Once cured, AgNWs were embedded just below the PDMS surface to form an AgNW/PDMS electrode. A commercial cable (PALS® Electrodes, Axelgaard) was attached to the AgNW/PDMS customized electrode to connect it to the current-controlled stimulator (Rehastim1, HASOMED GmbH). FIG. 1A illustrates the AgNW/PDMS customized FES electrode, and FIG. 1B shows the schematic representation of the electrode's structure.

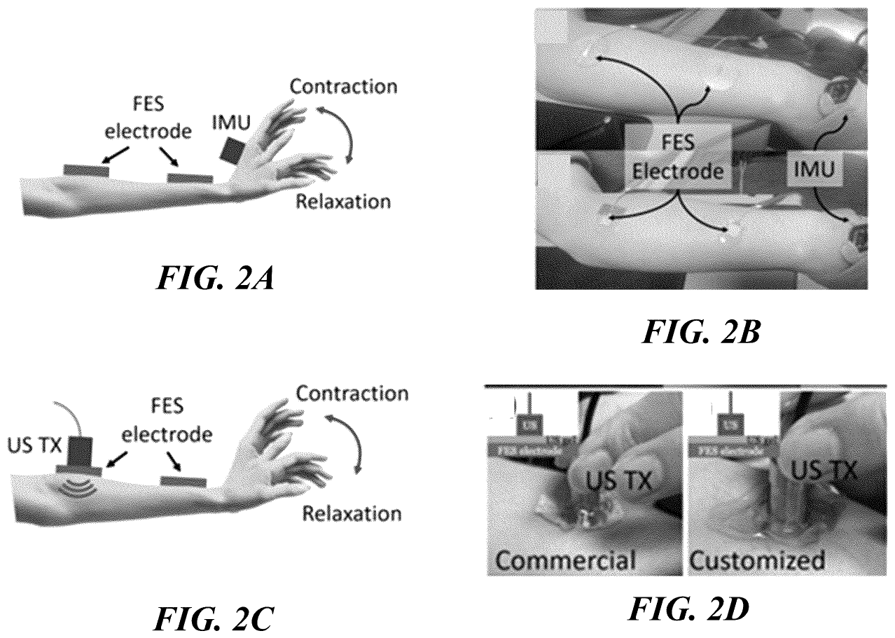

Evaluation of the Effectiveness of Ultrasound Compatible FES Electrode—A commercial hydrogel electrode (PALS® Electrodes, Axelgaard) was chosen for comparison study. To mitigate the potential impact of muscle fatigue induced by FES stimulation, a time interval was introduced between testing sessions involving the commercial electrode and AgNW/PDMS customized electrodes. This interval is a crucial consideration, as it allows for the dissipation of any residual muscle fatigue effects from the previous stimulation. By introducing this time gap, the study aims to ensure that each testing session commences with a relatively consistent baseline, thereby minimizing the potential effects of cumulative fatigue on the experimental outcomes. The experimental setup for the validation test is depicted schematically in FIG. 2A. The validation tests were conducted on the right forearm. The FES electrodes were placed atop the forearm, specifically at 5 cm from the lateral condyle, as well as the tendinous region, 5 cm from the carpals, as illustrated in FIG. 2B. Stimulation was applied to the forearm extensor to induce the wrist extension.

To quantify the wrist bend angle resulting from FES stimulation, an inertial measurement unit (IMU) was used. Relaxed status corresponded to a wrist bend angle of 0°. The angular change of the wrist, prompted by the FES stimulation delivered through the current-controlled stimulator (Rehastim1, HASOMED GmbH) was measured. This study aimed to assess the effects of distinct stimulation parameters, including current amplitude (5,8, and 11 mA), frequency (20, 30, and 40 Hz), and pulse duty cycle (20%, 40%, and 60%). Each FES stimulation lasted for 3 seconds and was repeated four times.

Experimental Setup—The schematic representation of the experimental setup for the feasibility test is illustrated in FIG. 2C. A 3.5 MHz single-element ultrasound transducer was employed in the feasibility test to estimate the insertion loss of both the customized and commercial electrodes. The single-element ultrasound transducer was placed directly on top of the electrodes in a vertical orientation to determine the acoustic compatibility of the customized electrode, as depicted in FIG. 2D. A pulser/receiver (5077 PR, Olympus, WA, USA) was connected to the transducer, which operated at a pulse repetition frequency (PRF) of 200 Hz and input voltage of 200 V. A bandpass filter was set within the range of 1 to 10 MHz. An oscilloscope (DSO7104B, Agilent Technologies, Santa Clara, CA, USA) was utilized to display the Radiofrequency (RF) signal.

For stimulation of the wrist extensor, the FES electrodes were placed on both the top of the forearm and the tendinous region. These electrodes were connected to a current-controlled stimulator. We selected parameters of 11 mA for current amplitude, 30 Hz for frequency, and 40% for the pulse duty cycle.

Initially, B-mode imaging of the target muscle was collected using a Clarius ultrasound probe (Vancouver, Canada) to establish a reference for the A-mode echo signal of the single-element ultrasound transducer. Time of flight (TOF) measurements were calculated and compared with B-mode imaging data. Subsequently, echo signals were obtained with and without the customized FES electrode to ascertain insertion loss. Lastly, the single-element transducer was positioned above both the customized and commercial electrodes to evaluate the feasibility of collecting echo signals.

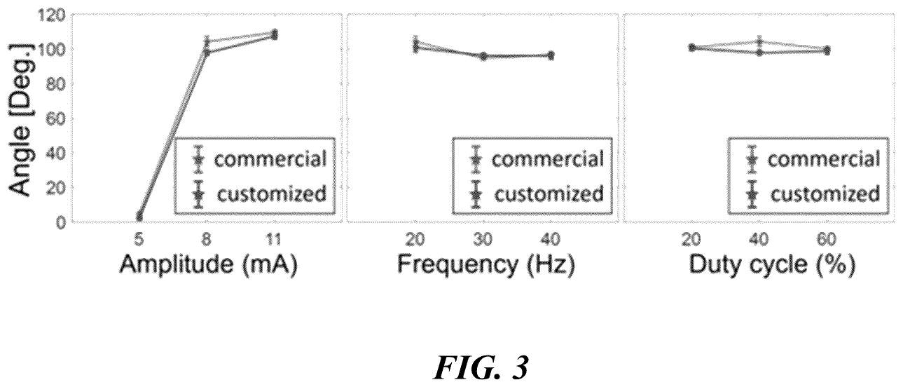

Results—Effectiveness of Ultrasound Compatible FES Electrode—To evaluate the effectiveness of the customized FES electrode, a comparative test was conducted against the commercial FES electrode. FIG. 3 shows the electrical stimulation effect of the customized FES electrode on the wrist extensor, juxtaposed with the performance of the commercial FES electrode. Various stimulation parameters: current amplitude, frequency, and pulse duty cycle of electrical stimulation were adjusted for the electrical stimulation. Notably, 5 mA of current amplitude was found to be the sub-threshold for both electrodes. In terms of performance, the customized FES electrode exhibited a comparably minor variance (<8%) relative to the commercial hydrogel electrode.

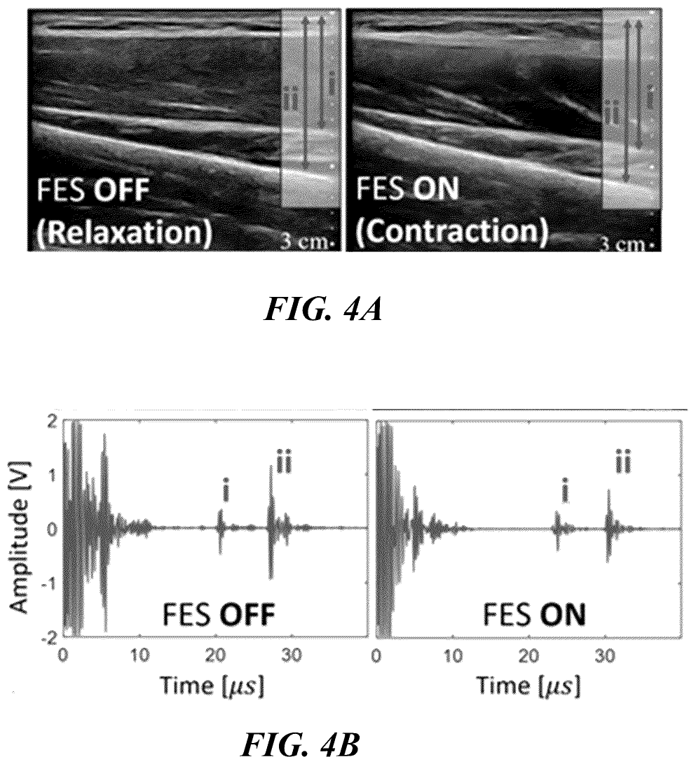

Feasibility of Ultrasound Compatible FES Electrode—Initially, B-mode imaging was captured as a reference for the A-mode ultrasound signals acquired using a single-element ultrasound transducer. FIG. 4A illustrates ultrasound imaging of the target muscle during states of relaxation and contraction of the wrist extensor. The muscle layers (labeled i and ii) constitute the regions of interest. In the relaxed state, these layers of muscle were situated approximately 1.5 cm and 2.1 cm beneath the skin surface, respectively. Following FES stimulation, the layers moved around 1.8 cm and 2.3 cm deeper from the skin, respectively. In FIG. 4B, the first echo occurred at 20.6 μs, corresponding to a distance of 1.58 cm, as calculated using time of flight. During muscle contraction, the subsequent echo at 27.25 μs corresponds to 2.1 cm in the relaxed state. During muscle contraction, the echo (labeled i) appeared at 23.75 μs, signifying a depth of 1.83 cm. The subsequent echo (labeled ii) at 30.3 μs indicated a distance of 2.3 cm, closely aligned with the location of muscle layers observed in B-mode imaging.

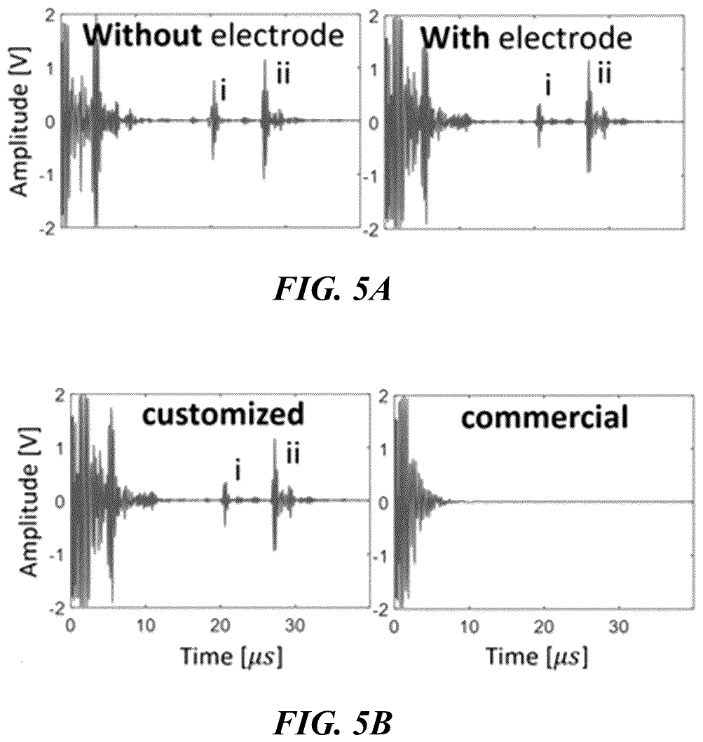

Next, to see the insertion loss of the customized FES electrode, changes in peak-to-peak voltage (Vpp) were measured based on the presence or absence of the customized FES electrode, as shown in FIG. 5A. Without the customized FES electrode, the Vpp values of the first and second echo signals (labeled i and ii) were measured to be 1.48 V and 2.23 V, respectively. When the customized FES electrode was positioned vertically on the front of the transducer, the Vpp value of the first and second echoes (labeled i and ii) from the muscle layers were recorded as 0.83 V and 2.1 V. Although the first echo experienced a reduction of 44%, the following echo decreased by 6% in comparison to the Vpp measurement taken without the customized FES electrode.

Finally, the customized electrode was compared with the commercial FES electrode. As shown in FIG. 5B, the echo signal from the muscle layers was successfully captured in the presence of the customized electrode. However, the commercial electrode cannot yield an ultrasound signal from the muscle layers. The findings demonstrated the customized electrode was compatible with ultrasound, allowing the transducer to effectively capture signals in the area of FES stimulation.

Discussion and Conclusion—The tests unveiled that the AgNW/PDMS customized electrode can be ultrasound compatible. Unlike commercial FES electrodes, PDMS on the customized FES electrode effectively shielded the EMI when the ultrasound transducer was vertically positioned on the electrode. The study applied Tensive® conductive adhesive gel (Parker Laboratories, Inc., New Jersey) on the AgNW side of the electrode. Although the gel helped to secure the electrode to the target area, its application was messy and was difficult to clean post-experiment. It also irritated the patients' skin upon removal.

The rigid single-element ultrasound transducer should require exertion of pressure to elicit echo signals from the target muscle even amidst FES stimulation. The pressure on the skin could potentially affect the alignment of the FES electrodes with the intended stimulated area, despite the usage of the adhesive gel. Referring to FIG. 4B, the reduction in peak-to-peak voltage by 44% for the first echo and 6% for the second echo could be attributed to the alignment of the transducer with each muscle layer. Thus, this highlights the potential necessity for a wearable single-element ultrasound transducer and arrays that not only eliminate the need for pressure on the transducer to collect data, but also mitigate concerns regarding misplacement of both the FES electrode and the ultrasound transducer.

This study introduced an AgNW/PDMS customized FES electrode designed to be ultrasound compatible, and the effectiveness of the electrical stimulation electrode was evaluated across various stimulation parameters. The assessment revealed a performance akin to that of the commercial hydrogel electrodes. Importantly, the customized FES electrode, positioned vertically beneath the transducer, was acoustically matched so that the ultrasound transducer could capture the echo signal from the forearm muscle layers with minimal insertion loss. The findings strongly suggest the promising prospects of an ultrasound-compatible FES electrode, enabling precise monitoring of muscle activities in the intended region targeted by FES stimulation.

### Example System and Study #2—Development of a Wearable Ultrasound Transducer for Sensing Muscle Activities in Assistive Robotics Applications: In Vitro Study

Robotic prostheses and powered exoskeletons are novel assistive robotic devices for modern medicine. Muscle activity sensing plays an important role in controlling assistive robotics devices. Most devices measure the surface electromyography (sEMG) signal for myoelectric control. However, sEMG is an integrated signal from muscle activities. It is difficult to sense muscle movements in specific small regions, particularly at different depths. Alternatively, traditional ultrasound imaging has recently been proposed to monitor muscle activity due to its ability to directly visualize superficial and at-depth muscles. Despite their advantages, traditional ultrasound probes lack wearability. In this study, a wearable ultrasound (US) transducer, based on lead zirconate titanate (PZT) and a polyimide substrate, was developed for a muscle activity sensing demonstration. The fabricated PZT-5A elements were arranged into a 4×4 array and then packaged in polydimethylsiloxane (PDMS). In-vitro pork experiments were carried out by generating muscle activities artificially, and the muscle movements were detected by the proposed wearable US transducer via muscle movement imaging. Experimental results showed that all 16 elements had very similar acoustic behaviors: the averaged central frequency, −6 dB bandwidth, and electrical impedance in water were 10.59 MHz, 37.69%, and 78.41Ω, respectively. The in vitro study successfully demonstrated the capability of monitoring local muscle activity using the prototyped wearable transducer. The findings indicate that ultrasonic sensing may be an alternative to standardizing myoelectric control for assistive robotics applications.

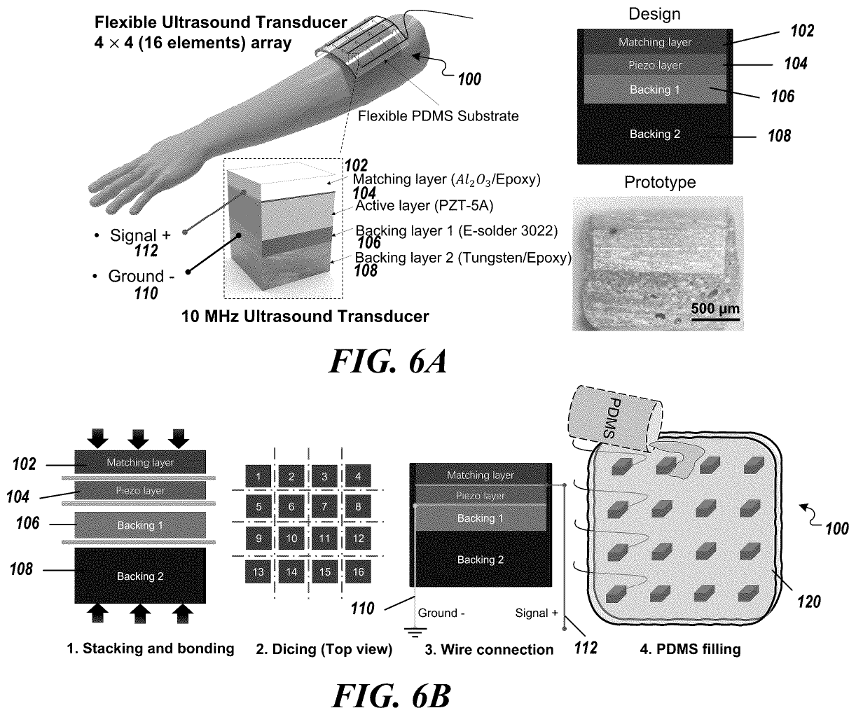

Transducer design and fabrication—A schematic demonstration of the wearable ultrasound (US) transducer 100 is shown in FIG. 6A. The wearable US array 100 was designed to have 16 elements in this study. Each element was individually wire connected to a coaxial cable to reduce the potential wire damage during any bending or extended motion. Due to the depth of penetration required for muscle movement detection, each element had a center frequency of 10 MHz. The 10 MHz elements consisted of one PZT-5A piezo ceramic plate (lateral size: 1.4 mm and thickness: 0.2 mm) which was diced mechanically and lapped down to obtain the desired thickness. An acoustic matching layer 102 (thickness: 0.25 mm) made of aluminum oxide/epoxy (particle size: 50 nm) was attached to the lapped piezo layer 104 by epoxy (EpoTek 301, Epoxy Tech. 107 Inc., San Jose, CA, USA). The electrically conductive epoxy (E-Solder 3022, Von-Roll Inc., Cleveland, OH, USA) was attached to the back side of the piezo layer 104 as the first backing layer 106 (thickness: 0.28 mm). Additionally, the mixture of epoxy with tungsten particles was cast to the first backing layer 106 as a second backing layer 108.

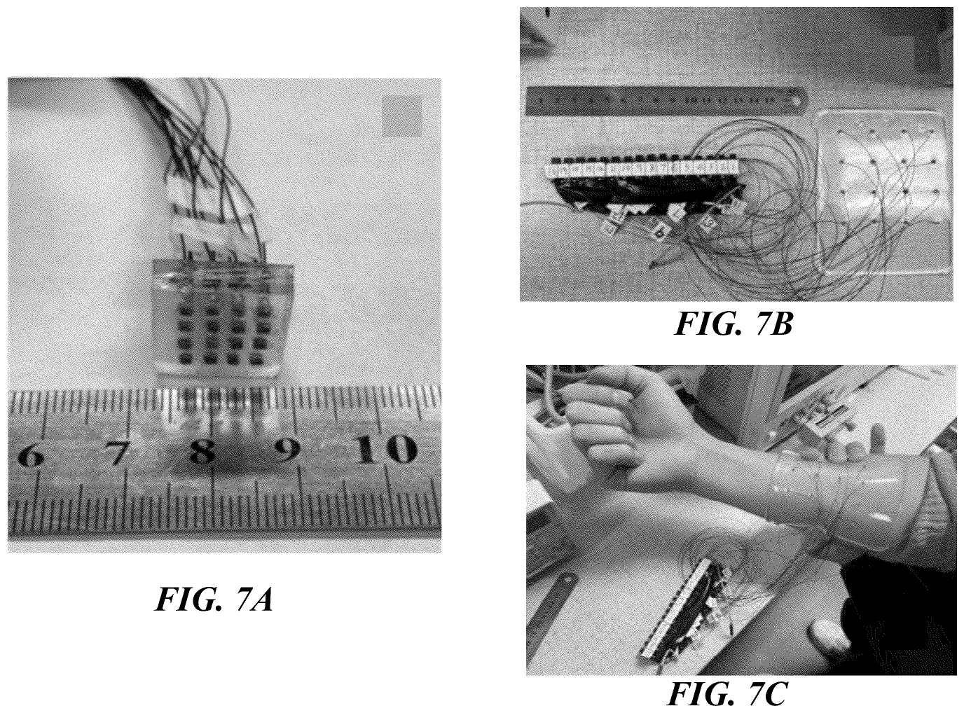

The fabrication process of the wearable US transducer was mainly divided into four steps, as shown in FIG. 6B. First, the matching layer 102, piezo layer 104, silver/epoxy backing layer 106, and tungsten/epoxy backing layer 108 were stacked and bonded with EpoTek 301 epoxy. Second, the bonded stacks were diced into individual small elements (lateral size: 1.4 mm). Third, the coaxial cable was connected to each element. The ground connection 110 was implemented onto the electrode placed on the backside of the piezo layer 104 with an E-Solder 3022 epoxy. The positive cable 112 was carefully bonded to the electrode of the front side of the piezo layer 104 with conductive epoxy. After the wire connection, the elements were coated with parylene-C (SCS Labcoter, PDS 2010, SCS, Indianapolis, IN) to provide a protective layer. Finally, the fabricated elements were attached to the square-shaped 3D printed mold (length: 100 mm, width: 80 mm, and height: 3 mm) and arranged into a 4×4 array with about 18 mm between each element. The PDMS was then poured into the 3D printed mold to form the flexible transducer array substrate 120. Subsequently, the PDMS-filled mold was transferred to the oven and kept at a temperature of 50° C. for 6 hours to be fully cured. The photographs of the proposed transducers are shown in FIGS. 7A-7C, according to various implementations.

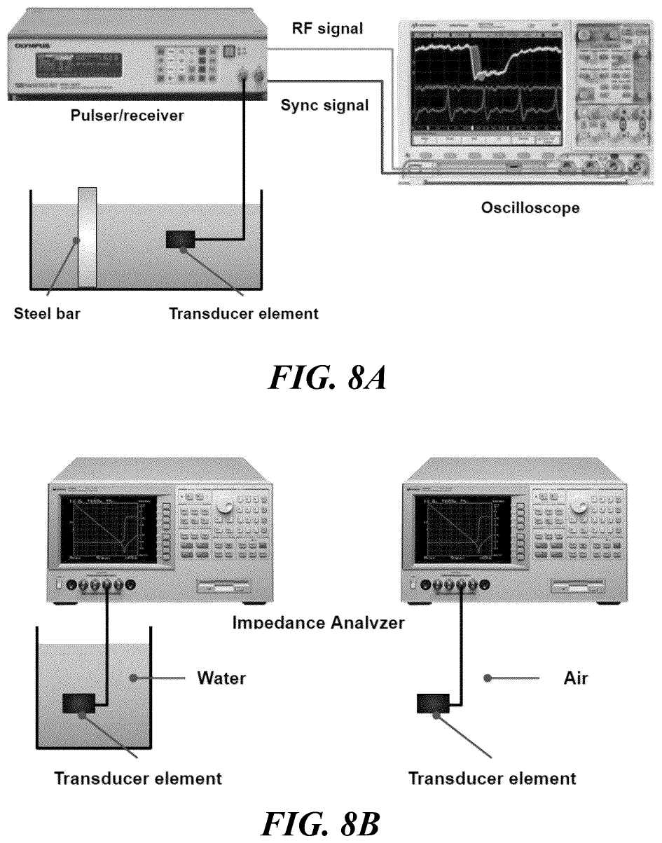

Transducer Characterizations—First, a pulse-echo test of the transducer was performed to evaluate the bandwidth and central frequency of all 16 fabricated elements, as shown in FIG. 8A. A pulser/receiver (5900 PR, Olympus, WA, USA) with a pulse repetition frequency (PRF) of 200 Hz and pulse energy of 1 μJ was used to excite the elements. A bandpass filter of 3 to 20 MHz was set for receiving the echo signals. A steel bar served as the reflector. The Radio-frequency (RF) signal was captured via an oscilloscope (DSO7104B, Agilent Technologies, Santa Clara, CA, USA). The bandwidth and central frequency of the fabricated elements were determined from the measured pulse-echo signal. To determine the electric impedance, capacitance, and loss, elements were connected to the impedance analyzer (4294A, Keysight Technologies Santa Rosa, CA, USA) and measured in the air and water separately. FIG. 8B illustrates the setup for electrical impedance measurements.

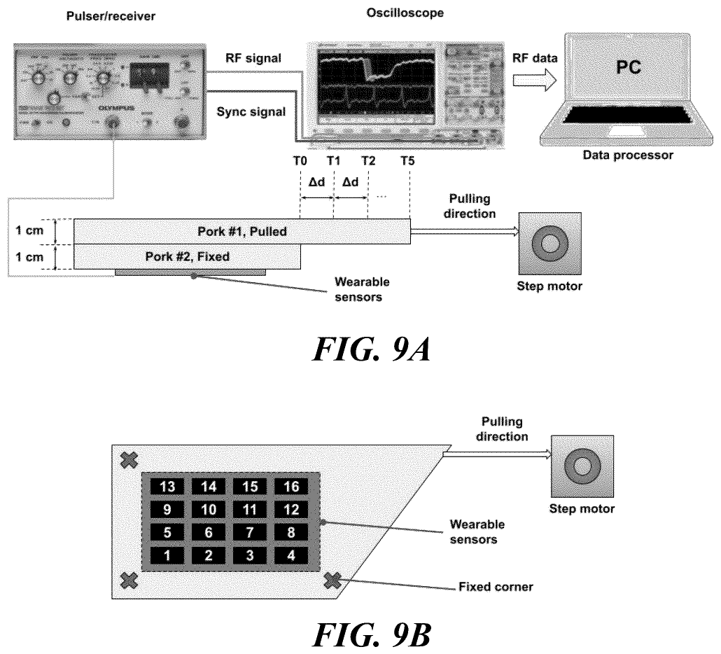

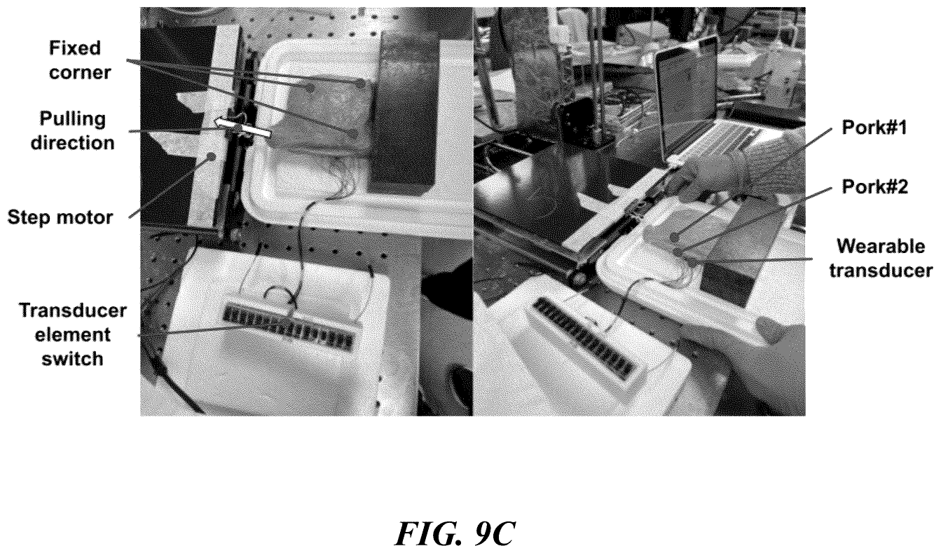

In vitro Experimental Setup—In-vitro pork muscle experiments were carried out to simulate muscle motions to demonstrate the performance of the wearable US device. The block diagram of the experimental system setup is shown in FIG. 9A. From top to bottom, pork #1 and pork #2 with a thickness of 1 cm were placed on the transducer, as shown in FIG. 9A (side view). Both transducer and pork #2 were fixed with pins, while pork #1 was fixed at three corners. A step motor was used to apply a pulling force to the non-fixed corner of pork #1, as shown in FIG. 9B (top view). A pulser/receiver (5077 PR, Olympus, WA, USA) with a PRF of 200 Hz, a pulse energy of 2 μJ, a gain of 20 dB, and a high pass filter of 1 MHz, was used to excite the transducer while pork #1 was pulled. One corner of pork #1 was pulled by the step motor with five equal steps over a distance of 5 mm during the experiments. An oscilloscope (DSO7104B, Agilent Technologies, Santa Clara, CA, USA) was used to capture the RF signal from all 16 elements as pork #1 was pulling from T0 to T5 (T0 represents the RF signal before pulling, the distance between time intervals is 1 mm). FIG. 9C shows the photos for the experimental setup. The sound velocity in a muscle tissue is typically about 1550 m/s. Since the thicknesses of both pork #1 and pork #2 are 1 cm, the time intervals between pork #1 and #2 were determined from the backscattering signals of muscles to separate different muscles.

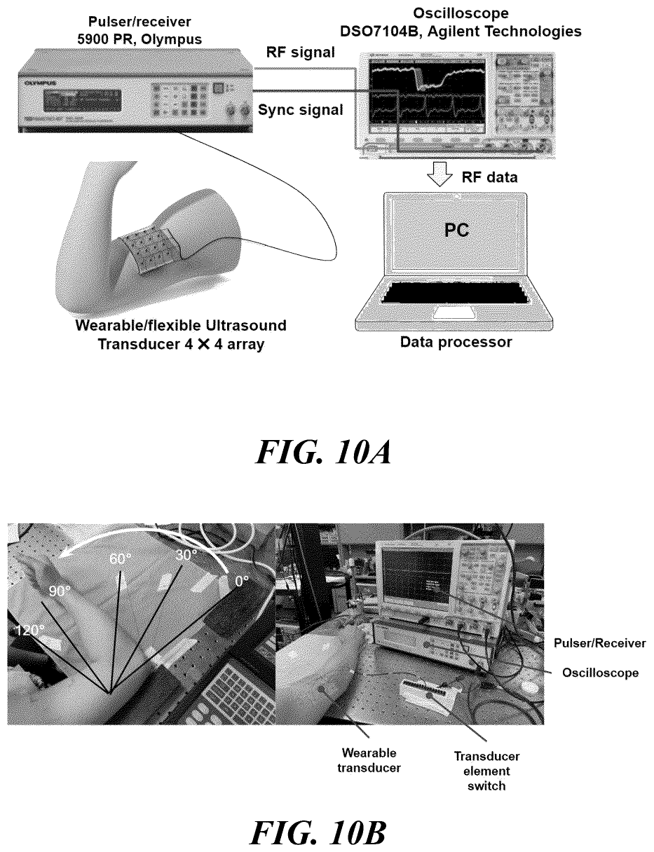

Preliminary In-vivo Experimental Setup—A preliminary in-vivo experiment was conducted to demonstrate the performance of using the proposed wearable transducer for detecting muscle activity in humans. FIG. 10A illustrates the block diagram of the experimental system setup. As shown in FIG. 10B, wearable transducers were attached to the subject's biceps brachii muscle to detect muscle activity as the subject flexed his elbow from 0° to 120°. Three trials were conducted in total. A pulser/receiver (5900 PR, Olympus, WA, USA) with a pulse energy of 1 μJ, a gain of 40 dB, a high pass filter of 3 MHz, and a low pass filter of 20 MHz, was used to excite the transducer while the forearm was moved. An oscilloscope (DSO7104B, Agilent Technologies, Santa Clara, CA, USA) was used to capture the RF signal from all 16 elements as the subject flexed his elbow from 0° to 120° with 30° intervals. The photographs for the experimental setup are shown in FIG. 10B.

RF Data Processing Procedure—The RF data of 16 elements were acquired from the oscilloscope with a sampling rate of 4 GHz for post-processing to detect the pork tissue displacements for the in-vitro test and human muscle motions for the preliminary in-vivo test of each element. All the signal and image processing was performed using MATLAB (R2020b, The Math Works, Natick, Massachusetts, USA). First, the CSV files recorded from the oscilloscope were converted to MATLAB files. After the files were loaded, the range (length) of muscle backscattering signals was determined according to the B-mode image from a commercial US system. The backscattering signal at the initial location was referred to as the reference signal (T0), and the tissue displacement was determined at each pulling step via the normalized cross-correlation (NCC), which calculated the difference in phase between the reference signal and the comparison signal:

$$N C C ( t ) = \frac{{\sum }_{n = 1}^{W} f ( n ) g ( n + t )}{\sqrt{{\sum }_{n = 1}^{W} {f}^{2} ( n ) \cdot {\sum }_{n = 1}^{W} {g}^{2} ( n + t )}} , ( {t}_{1} \leq t \leq {t}_{2} )$$

Where the f(n) and g(n) are the reference signal and comparison signal, respectively, n is the index of samples (from 1 to W), W is the window length of comparing samples, t is the time shift between the reference signal and the comparison signal, and [t1, t2] is the time range of interest in the reference signal. After the phase shifts of each element were determined at different pulling steps, the relative tissue displacements were then obtained since the angle between the US beam and muscle orientation was unknown. Subsequently, the relative muscle displacements were color encoded on a 4×4 matrix to show the muscle movement imaging. A linear interpolation method was applied to the matrix for image smoothing.

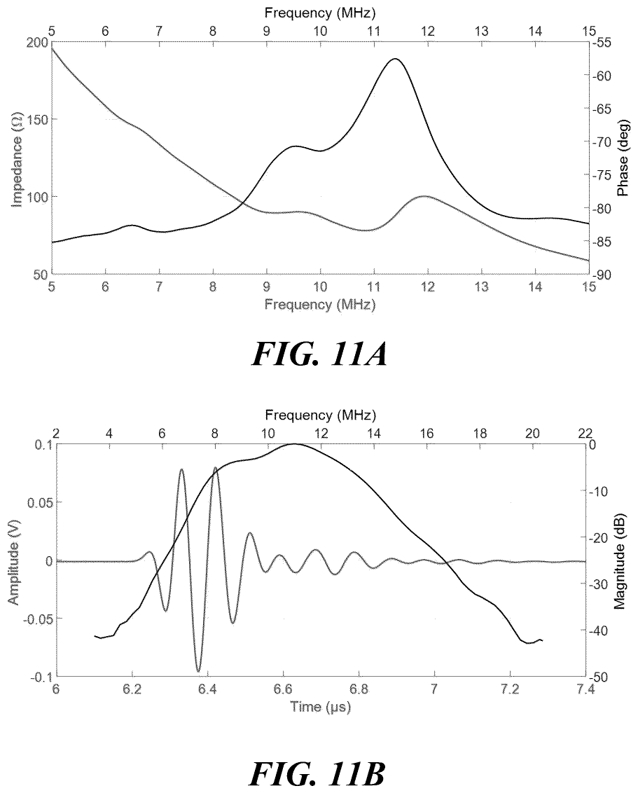

Results—Transducer Characterizations—FIG. 11A and 11B show the typically measured impedance of the transducer in the water and the pulse-echo results for one of the elements. Both measured results came from element #10 as a sample. FIG. 11A depicts that the central frequency was 10.61 MHz with an impedance of 75.27Ω in the water. Capacitance and dielectric loss were 176.98 pF and 9.37 mU, respectively. FIG. 11B shows the pulse-echo result for element #10. With 1 μJ pulse energy, the peak-to-peak amplitude was 175.8 mV, and the −6 dB bandwidth was 48.82%. Furthermore, the characterizations of all 16 elements are listed in Table 1, below.

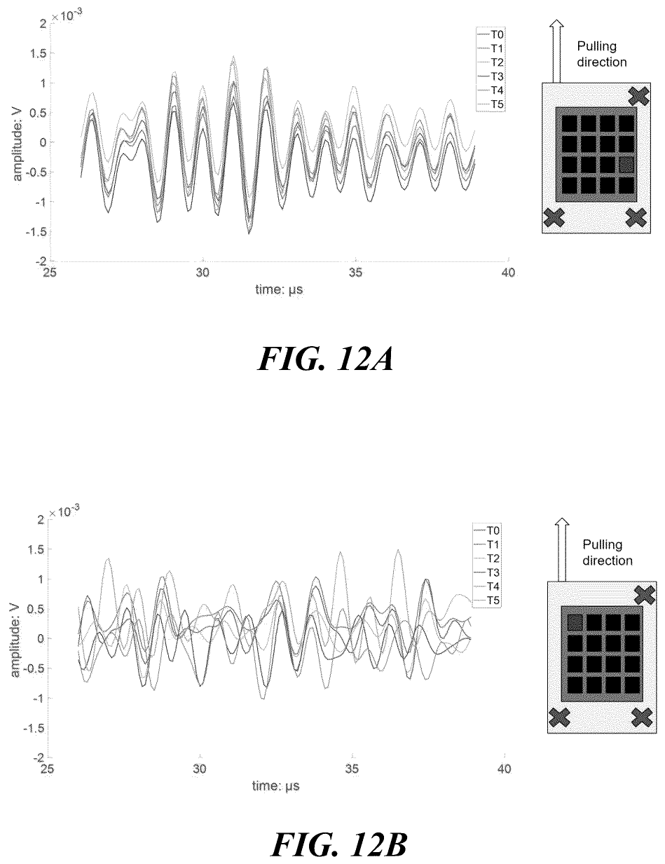

In-vitro results—FIG. 12A shows the typical ultrasonic backscattering signals of pork at element #2 from different pulling distances (from T0 to T5). Since the pork muscles were fixed at three corners surrounding element #2, muscle displacement was limited during pulling. Therefore, it is obvious that the phases of ultrasonics backscattering signals at element #2 seem similar during pulling, as shown in FIG. 12A. On the contrary, larger variations of ultrasonic backscattering signals were observed clearly at element #16 from different pulling distances (T0 to T5), as shown in FIG. 12B, where the location of element #16 was close to the pulling site that exhibited a larger muscle displacement. In other words, the muscle displacements were detected regionally by all 16 elements of the wearable US transducer.

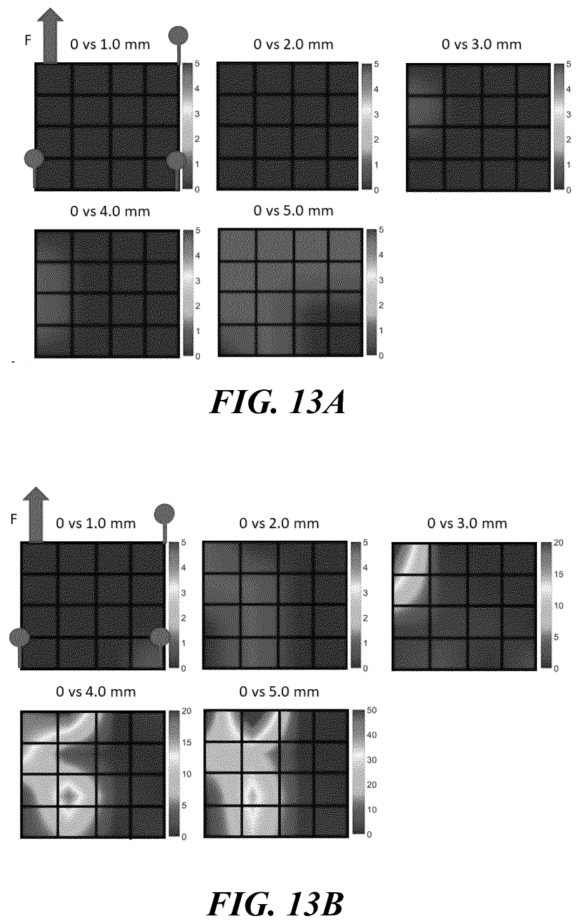

FIG. 13A shows the typical muscle movement imaging from pork #2 at different pulling distances. The relative muscle displacements were obtained by calculating the phase change of backscattering signals during pulling via the NCC method. The 4×4 matrix represents the locations of 16 elements. Since pork #2 was fixed without pulling, the relative muscle displacements seem to remain constant for all elements during the experiment. On the contrary, according to FIG. 13B the muscle movement imaging from pork #1 exhibited a larger variation of relative displacement during pulling. Muscle displacements were increased with pulling, especially around the pulling site, which is in line with the experimental setup in that only pork #1 was pulled during the experiment. This experimental result is evidence that the proposed wearable transducer can not only detect regional muscle displacements but also at different depths.

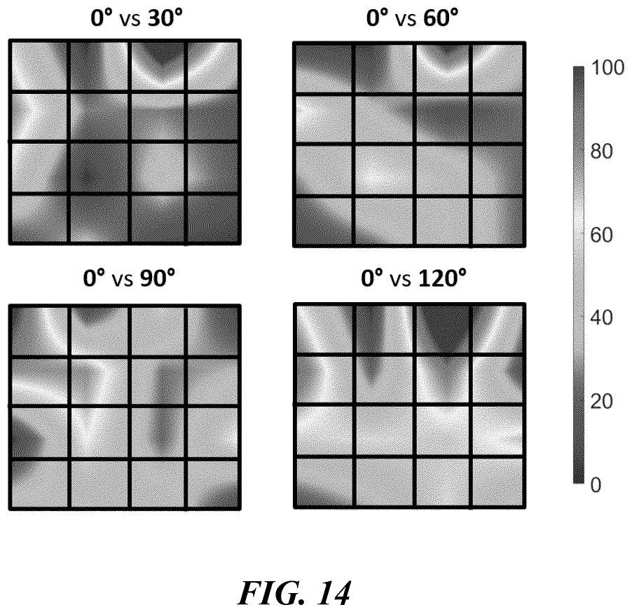

Preliminary In-vivo Results—FIG. 14 illustrates the typical muscle movement images of three trials of the biceps brachii at various elbow flexion angles (average from three trials). In a similar manner to the in-vitro results, the relative muscle displacements were determined by calculating the phase change of backscattering signals during forearm movement using the NCC method. The muscle displacements were obtained from the proposed wearable transducer during body movement. Additionally, there was a larger displacement in the region of the target muscle associated with the bending movement, which is represented in the color map as red. It is possible to determine the role of a specific muscle using this method.

TABLE 1
Acoustic Characterizations of 16 Elements

| | Pulse-echo response test | | | Electrical impedance test | | | |
|---|---|---|---|---|---|---|---|
| | Central | Fractional | | Capacitance | Loss | Impedance | Impedance |
| | frequency | bandwidth | Loop | (@ 1 kHz, | (@ 1 kHz, | in Air | in Water |
| Property | (MHz) | (−6 dB) % | Sensitivity | pF) | pF) | (Ω) | (Ω) |
| Element #1 | 10.04 | 37.05 | −39.28 | 199.00 | 9.80 | 79.06 | 78.30 |
| Element #2 | 10.71 | 30.44 | −49.82 | 186.50 | 9.90 | 80.53 | 81.35 |
| Element #3 | 10.69 | 21.89 | −39.05 | 199.80 | 9.77 | 76.09 | 72.50 |
| Element #4 | 9.97 | 59.18 | −50.27 | 189.70 | 9.37 | 79.64 | 80.83 |
| Element #5 | 10.61 | 32.61 | −38.87 | 194.68 | 10.55 | 79.17 | 79.04 |
| Element #6 | 10.82 | 29.57 | −37.42 | 196.27 | 9.70 | 75.07 | 76.23 |
| Element #7 | 10.37 | 45.71 | −45.96 | 193.89 | 10.90 | 81.04 | 86.28 |
| Element #8 | 10.85 | 32.26 | −40.74 | 191.54 | 9.70 | 76.50 | 75.46 |
| Element #9 | 10.85 | 36.31 | −35.09 | 199.46 | 10.00 | 76.19 | 73.00 |
| Element #10 | 10.61 | 48.82 | −38.02 | 176.98 | 9.37 | 77.94 | 75.27 |
| Element #11 | 10.18 | 40.47 | −37.27 | 194.72 | 10.64 | 74.85 | 75.70 |
| Element #12 | 10.97 | 30.81 | −40.47 | 199.50 | 10.20 | 76.92 | 77.75 |
| Element #13 | 10.83 | 27.52 | −35.83 | 191.45 | 10.10 | 79.60 | 80.00 |
| Element #14 | 10.81 | 32.75 | −39.42 | 184.60 | 9.90 | 85.88 | 80.43 |
| Element #15 | 10.93 | 33.12 | −37.18 | 184.80 | 10.30 | 85.69 | 79.65 |
| Element #16 | 10.16 | 64.57 | −39.42 | 180.10 | 9.10 | 83.36 | 82.81 |
| Average | 10.59 | 37.69 | −40.26 | 191.44 | 9.96 | 79.22 | 78.41 |
| Standard | 0.33 | 11.55 | 4.53 | 7.13 | 0.48 | 3.45 | 3.65 |
| Deviation | | | | | | | |

Discussion—This study described the development of a wearable US device in which the PZT-5A elements are embedded into deformable PDMS substrates. The rigidity of PZT led to the selection of PDMS as a flexible substrate for providing mechanical interlinkage between elements. In contrast to traditional US transducers, which are based on rigid substrates, the PDMS substrate is selected for several reasons. To begin with, PDMS has a stretchability of >170% in tensile strain. In addition to its flexibility, as PDMS is biocompatible, it can be safely applied in biomedical applications in close contact with the skin. Besides, PDMS material is thermally secured at certain high temperatures over 200° C., which means it is safe to use it for ultrasound transducers. PDMS has suitable electrical and acoustic properties for the ultrasound transducer. It could be essential to determine the geometric design of the flexible ultrasound transducer such as the spacing between adjacent elements, the aperture size of each element, the number of elements, and so forth. Otherwise, each element could have a constructive effect on the reduced performance of the flexible array transducer when it is deformed. There may be a concern regarding the dissipation of the acoustic stack with PZT from PDMS. However, the proposed flexible ultrasound transducer array is fabricated to detect muscle movement signals. By having the large spacing between each element corresponding to target muscles, it may be possible to prevent detachment issues due to the relatively lower strain applied to the transducer while it is being deformed. Furthermore, the wearable transducer is relatively thin, with a thickness of approximately 3 mm. The wires of each element are individually connected with a coaxial cable to reduce the possibility of wire damage during bending or extended motion. As a result, the device is highly stretchable and reliable.

The conventional handheld ultrasonic transducer with a rigid housing has limits in its ability to detect muscle activity as the target muscle (upper or lower limbs) is moving dramatically. When an ultrasonic transducer is pressed against a body surface, it may inhibit the activity of underlying muscles. Additionally, the accuracy of the measurement of muscle activity may be influenced significantly as the surface of a conventional transducer does not attach to the skin very well. In contrast, the flexible and wearable transducer can attach to the body area of interest without restricting the movement of the underlying tissue and preventing transducer shifting. Furthermore, the sizes of PDMS substrates, the thickness of the piezo layer, and transducer arrangements can be easily tailored to fit the location, depth, and shape of the target muscle. The wearable transducer developed in the present study is capable of accurately monitoring muscle activity. Since the muscle activities can be detected regionally by the wearable transducer, ideas for how to use these US signals to control the robotic prosthesis will be the next works. In addition, customizable devices make it possible to measure multiple muscle groups simultaneously and individually, which is an important step in developing control schemes for high-degree-of-freedom assistive robotics. This study confirms that the wearable device is capable of monitoring muscle movement. Quantitative comparisons between non-wearable US transducers and the proposed wearable US system are planned. Moreover, over thirty tests have been conducted for in vitro and in vivo experiments using the proposed flexible US transducer array without interruption in performance due to heat generation or dissipation.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A wearable ultrasound sensor configured to detect muscle activities, the wearable ultrasound sensor comprising:
an array of ultrasound transducers, wherein each ultrasound transducer comprises:
a matching layer on a top side;
an active layer adjacent the matching layer;
a first backing layer adjacent the active layer;
a second backing layer adjacent the first backing layer; and
an epoxy bonding material interspaced between each of the matching layer, the active layer, the first backing layer, and the second backing layer,
wherein the array of ultrasound transducers is surrounded by a PDMS filling,
whereby the active layer is configured to communicate a signal in response to mechanical deformation.

2. The wearable ultrasound sensor of claim 1, wherein the wearable ultrasound sensor is also configured as a wearable functional electrical stimulation (FES) electrode.

3. The wearable ultrasound sensor of claim 1, wherein the matching layer comprises aluminum oxide and epoxy.

4. The wearable ultrasound sensor of claim 1, wherein the active layer is a piezo layer comprising PZT-5A.

5. The wearable ultrasound sensor of claim 1, wherein the first backing layer comprises E-solder 3022 and the second backing layer comprises tungsten and epoxy.

6. The wearable ultrasound sensor of claim 1, further comprising a wire connection to each of a top side of the active layer and a bottom side of the active layer.

7. The wearable ultrasound sensor of claim 1, wherein the array of ultrasound transducers comprises 16 ultrasound transducers.

8. The wearable ultrasound sensor of claim 1, further comprising a data acquisition system in electrical communication with each of the ultrasound transducers of the array of ultrasound transducers, the data acquisition system configured to collect data from the array of ultrasound transducers during a muscle activation or muscle detection operation.

9. The wearable ultrasound sensor of claim 8, further comprising a controller in electrical communication with the data acquisition system, the controller configured for processing signals from the array of ultrasound transducers.

10. The wearable ultrasound sensor of claim 1, wherein the array of ultrasound transducers is configured to be coupled to at least a portion of a muscle grouping of a user.

11. The wearable ultrasound sensor of claim 1, wherein a thickness of each ultrasound transducer is less than 1.5 mm.

12. The wearable ultrasound sensor of claim 1, wherein the array of ultrasound transducers has a center frequency of 10 MHz configured to detect muscle movements.

13. A system comprising:
a wearable ultrasound sensor configured to detect muscle activities, the wearable ultrasound sensor comprising:
an array of ultrasound transducers, wherein each ultrasound transducer comprises:
a matching layer on a top side;
an active layer adjacent the matching layer;
a first backing layer adjacent the active layer;
a second backing layer adjacent the first backing layer; and
an epoxy bonding material interspaced between each of the matching layer, the active layer, the first backing layer, and the second backing layer,
wherein the array of ultrasound transducers is surrounded by a PDMS filling, and
whereby the active layer is configured to communicate a signal in response to mechanical deformation; and
a controller in electrical communication with the array of ultrasound transducers of the wearable ultrasound sensor, the controller configured to collect and interpret signals from the array of ultrasound transducers in response to a muscle activity.

14. The system of claim 13, wherein the active layer of the wearable ultrasound sensor is a piezo layer comprising PZT-5A, the active layer configured to send a signal to the controller in response to mechanical deformation.

15. The system of claim 13, wherein the wearable ultrasound sensor is also configured as a wearable functional electrical stimulation (FES) electrode, and the controller is configured to send electrical stimulation signals to the array of transducers of the wearable ultrasound sensor.

16. A method of fabricating a wearable ultrasound sensor, the method comprising:
providing a matching layer, an active layer, a two backing layers;
stacking the matching layer, the active layer, and the two backing layers using an epoxy to form a bonded stack;
dicing the bonded stack into a plurality of ultrasound sensors including a first ultrasound sensor;
coupling a wire to the first ultrasound sensor, the wire configured to relay data from the first ultrasound sensor to a data acquisition system or a controller; and
coating the plurality of ultrasound sensors with a protective material which, when cured, forms a flexible substrate within which the plurality of ultrasound sensors is arranged in an array.

17. The method of claim 16, wherein the wearable ultrasound sensor is configured for conforming and attaching to a skin surface of a mammal.

18. The method of claim 16, wherein the protective material is liquid PDMS that cures to form the flexible substrate.

19. The method of claim 16, wherein the first ultrasound sensor of the plurality of ultrasound sensors has a width less than 1.5 mm and a thickness less than 1.5 mm.

20. The method of claim 16, further comprising:
providing a controller coupled to and in electrical communication with the plurality of ultrasound sensors, the controller configured to collect muscle movement signals from the plurality of ultrasound sensors.
